(19) United States
(12) Patent Application Publication
(10) Pub. No.: US 2024/0334965 A1
(43) Pub. Date: Oct. 10, 2024

(54) System and Process for Manufacturing Cannabis Joints

(71) Applicant: Le Groupe Solid Packaging Robotik Inc., Terrebonne (CA)

(72) Inventors: Robert Archambault, Boisbriand (CA); Daniel Martel, Terrebonne (CA); Harold Bouchard, Bois-des-Filion (CA)

(21) Appl. No.: 18/295,583

(22) Filed: Apr. 4, 2023

(51) Int. Cl. A24C 5/32; A24C 5/12; A24C 5/60; A61K 36/185; B65G 47/90

(52) U.S. Cl. CPC A24C 5/322; A24C 5/12; A24C 5/605; A24C 5/608; A61K 36/185; B65G 47/90

(57) ABSTRACT

A cannabis joints manufacturing machine includes a plurality of cannabis joints manufacturing devices positioned in a line. The first of these cannabis joints manufacturing devices is configured to receive a cannabis cone including cannabis therein, and each of the other stations are adapted to perform on it a step among a sequence of steps towards manufacturing a cannabis joint. The last one of the plurality of cannabis joints manufacturing devices transforms the cannabis cone resulting from the processing by the previous stations into a cannabis joint. The cannabis joints manufacturing machine further includes a cannabis cone moving device for simultaneously moving the cannabis cones in the plurality of cannabis joints manufacturing devices to a next cannabis joints manufacturing device in the line.

### FIELD

The present disclosure generally relates to cannabis joints and more specifically to cannabis joint manufacturing systems and processes.

### BACKGROUND

Cannabis joints have long been and are still very often prepared by hands.

However, the legalization of cannabis in many territories have seen the coming of the automatization in the manufacturing of cannabis joints.

Some of the automated processes in the manufacturing of cannabis joints involve inserting paper cones in a cone holding tray and then vibrating the whole tray while it receives cannabis in bulk, the vibrations aiming at evening out the filling of the cones.

A drawback of such vibrating tray is that strong and moderate vibrations have been found to separate the delta-9-tétrahydrocannabinol (THC) from cannabis, thereby decreasing the quality of the product.

Other known processes for manufacturing cannabis joints include moving together in parallel a series of cannabis cones between different stations, each performing a further step towards manufacturing the joints.

A drawback is that it renders tedious the quality control on each joint, considering that a plurality of cannabis joints is finished at a same time.

Also, the machines used to process many cannabis joints in parallel are bulky.

### SUMMARY

According to an illustrative embodiment, there is provided a cannabis products manufacturing system comprising:

a number S of at least two different cannabis products manufacturing stations, each for performing on one cannabis support at a time a step among a sequence of steps for manufacturing a cannabis product; the number S of at least two different cannabis products manufacturing stations being positioned side-by-side according to the sequence of steps for manufacturing the cannabis product; a first one of the cannabis products manufacturing stations being a cannabis support picking station for performing a first step among the sequence of steps, which is picking a single cannabis support among a plurality of cannabis supports; and

a cannabis support moving device, including a number S minus 1 of cannabis support grippers arranged side-by-side so that each of the cannabis support grippers is operatively paired with a corresponding one of the manufacturing stations for simultaneous reciprocal movement between the corresponding one of the manufacturing stations and a next one thereof; each of the cannabis support grippers being movable between a cannabis support holding position and a cannabis support releasing position.

According to another illustrative embodiment, there is provided a process for manufacturing cannabis products, the process comprising:
positioning side-by-side a number S of cannabis products manufacturing stations, each for performing, on a single one of a number S of cannabis supports at a time, a step among a sequence of steps for manufacturing a cannabis product; a first one of the number S of cannabis products manufacturing stations being a cannabis support picking station for performing a first step among the sequence of steps on a first one of the number S of cannabis supports, which is picking the first one of the number S of cannabis supports among a plurality of cannabis supports; a last step in the sequence of steps including outputting the last one of the number S of cannabis supports;
simultaneously operating each of the number S of cannabis products manufacturing stations on a respective one of the number S of cannabis supports; a last one of the number S of cannabis supports becoming the cannabis product; and
simultaneously moving each of a number S-1 of cannabis supports remaining in the number S of cannabis products manufacturing stations to a next one of the number S of cannabis products manufacturing stations;
repeating ii) and iii) for each remaining cannabis support among the number S of cannabis supports.

According to still another illustrative embodiment, there is provided a cannabis joints manufacturing machine comprising:
a plurality of cannabis joints manufacturing devices positioned in a line; a first one of the plurality of cannabis joints manufacturing devices receiving a cannabis cone including cannabis therein; each of the other one of the plurality of cannabis joints manufacturing devices being adapted to perform on the cannabis cone at least one step among a sequence of steps towards manufacturing a cannabis joint; a last one of the plurality of cannabis joints manufacturing devices transforming a cannabis cone therein into a cannabis joint and outputting the cannabis joint; and
a cannabis cone moving device for simultaneously moving the cannabis cones in the plurality of cannabis joints manufacturing devices to a next cannabis joints manufacturing device in the line.

According to still another illustrative embodiment, there is provided a cannabis products manufacturing system comprising:
a number S of at least two different cannabis joints manufacturing stations, each for performing on one cannabis cone at a time a step among a sequence of steps for manufacturing a cannabis joint; the at least two different cannabis joints manufacturing stations being positioned side-by-side according to the sequence of steps for manufacturing the cannabis joint; a first one of the cannabis joints manufacturing stations being a cannabis cone picking station for performing a first step among the sequence of steps, which is picking a single cannabis cone among a plurality of cannabis cones; and
a cannabis cone moving device, including a number S minus 1 of cannabis cone grippers arranged side-by-side so that each of the cannabis cone grippers is operatively paired with a corresponding one of the manufacturing stations for simultaneous reciprocal movement between the corresponding one of the manufacturing stations and a next one thereof; each of the cannabis cone grippers being movable between a cannabis cone holding position and a cannabis cone releasing position; wherein the cannabis support moving device includes a linear actuator comprising a base and a slider slidably mounted to the base; the number S minus 1 of cannabis support grippers being mounted to the slider;
wherein the number S of at least two different cannabis joints manufacturing stations are selected from the group consisting of a cannabis joint forming station and a cannabis oil injecting station.

It is to be noted that the term ‘station’ is to be construed herein as including any system, apparatus, machine and/or device that performs a specific task or step among a plurality of such tasks or steps required to achieve a result.

Also, the expression cannabis product is to be construed herein as including all type of product including cannabis in herb or similar form received in a container or provided on a support of any kind.

The expression cannabis support is to be construed herein as including the intrant to the cannabis manufacturing system described and claimed herein and its various transformed form through manufacturing stations/steps and up to the final product, which is the output product of the manufacturing system/method.

The term ‘step’ should be construed broadly herein as including one or more actions towards a specific result.

Other objects, advantages, and features of the process and system for manufacturing cannabis joints will become more apparent upon reading the following non-restrictive description of illustrative embodiments thereof, given by way of example only with reference to the accompanying drawings.

### BRIEF DESCRIPTION OF THE DRAWINGS

In the appended drawings:

FIG. 1 is a perspective view of a cannabis joint manufacturing system according to a first illustrative embodiment;

FIG. 2A is a perspective view of a cannabis joint picking station part of the system from FIG. 1;

FIGS. 2B and 2C are closeup perspective views of the picking station from FIG. 2A, illustrating the operation thereof;

FIG. 3 is a front elevation of the of the cannabis product forming station, and of the cannabis product moving device, both parts of the system from FIG. 1;

FIGS. 4A-4F are isolated perspective views of the station and device from FIG. 3; the station and device being shown through different steps of its operation; and

FIGS. 5A-5E are isolated perspective views of the cannabis oil injecting station, parts of the system from FIG. 1; the station being shown with the moving device through different steps of their operation;

FIG. 6 is a front elevation view of the system from FIG. 1; and

FIG. 7 is a flowchart illustrating a method for manufacturing cannabis joints according to an illustrative embodiment.

### DETAILED DESCRIPTION

In the following description, similar features in the drawings have been given similar reference numerals, and in order not to weigh down the figures, some elements are not referred to in some figures if they were already identified in a precedent figure. Herein, it shall further be noted that, for avoiding unnecessary details obscuring the invention, only device structures and/or processing steps closely relevant to schemes according to the invention are shown in the accompanying drawings while omitting other details less relevant to the invention.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, un-recited elements.

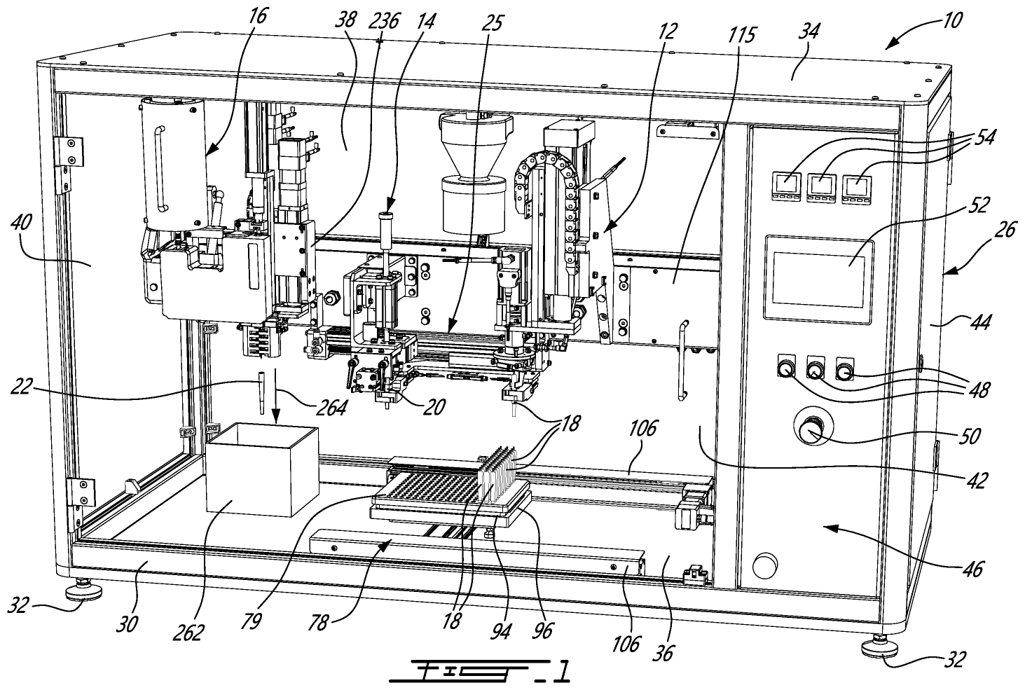

A system 10 for manufacturing cannabis products according to an illustrative embodiment will now be described with reference first to FIG. 1.

The system 10 comprises three (3) cannabis products manufacturing stations 12-16, each for performing on one cannabis support 18-20 at a time, a step among a sequence of steps for manufacturing a cannabis product 22, and a cannabis support moving device 25 for simultaneously moving the cannabis support 18-20 between stations 12-16. The stations 12-16 and moving device 25 are all included in a casing 26.

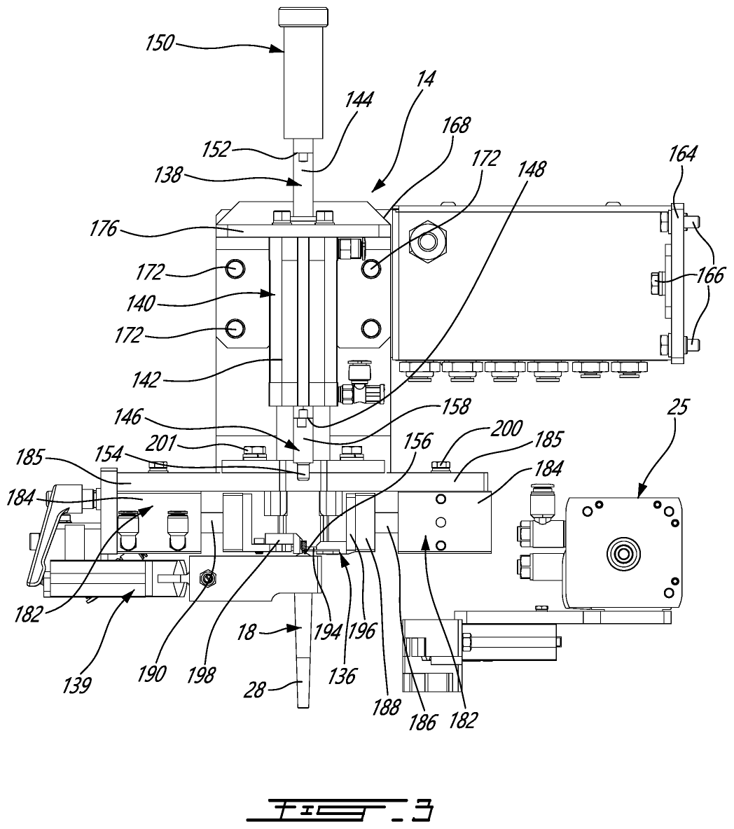

According to the illustrative embodiment, the cannabis supports 18-20 are, when inputted in the system, well-known cannabis cones 18 that have been prefilled with cannabis (not shown) and further provided with a filter 28 their bottom end (see on FIG. 3). These cannabis supports 18, are then transformed by the system from station 12-16 to station 12-16, resulting in a cannabis product, in the form of cannabis joints 22, whose characteristics will be described in more detail further in. As such, the expression cannabis support or cannabis cone is used herein and, in the claims, to refer to the intrant to the system 10, and to its various forms during different phases of its transformation by the system 10.

Also, it is to be noted that the expressions cone and joint are used herein to refer to the same product, but during different phase of its manufacture. More specifically, the expression “cone” will be generally used herein to refer to the product until the cannabis oil is injected therein, whereby, at that point, the product will be referred to as a ‘joint’. This terminology should however not be used in any restraining way.

Each of the components 12-16, 25 and 26 of the system 10 will now be described in further detail.

The casing 26 includes a generally cubic frame 30 mounted on four (4) adjustable legs 32 (only two shown), top, bottom, back and left side walls 34, 36, 38 and 40 respectively, and front and right-side doors 42 and 44, all mounted to the frame 30. The casing 26 further includes a control panel 46 on the front of the casing 26, extending between the front and right-side doors 42 and 44. The control panel 46 is provided with input and output devices in the form of control knobs 48 and 50 and display screens 52 and 54. The control panel 46 defines the front side of control module(s) (not shown) that are accessible from the right-side door 44 and acts as the interface thereto. The manufacturing stations 12-16 are also connected to the control module(s), which acts as their controller for their synchronized operations.

Since control modules and control panels for manufacturing stations are believed to be well-known in the art, they will not be described herein in more detail for concision purposes.

According to another illustrative embodiment, the legs 32 are replaced by wheels (not shown), rendering the system 10 mobile.

The manufacturing stations 12-16 and moving device 25 are secured to the cubic frame 30 and to the back wall 38.

The casing 26 defines an enclosure, which allows controlling the temperature therein, which can be beneficial for some types of intrants 18 and/or products 22 manufactured by the system 10. In such cases, temperature conditioning systems (not shown) are further provided in the system 10.

Illustrative embodiments of a cannabis products manufacturing system are not limited to include a casing as illustrated, which can have other configurations than illustrated herein or omitted.

Also, the manufacturing stations 12-16 and moving device 25 can be mounted to the casing 26 using any mounting elements or assemblies in addition to and/or alternatively to those that will be described hereinbelow in more detail.

According to the first illustrative embodiment, there are three (3) manufacturing stations 12-16:
a cannabis cone picking station 12;
a cannabis cone forming station 14; and
a cannabis oil injecting station 16.

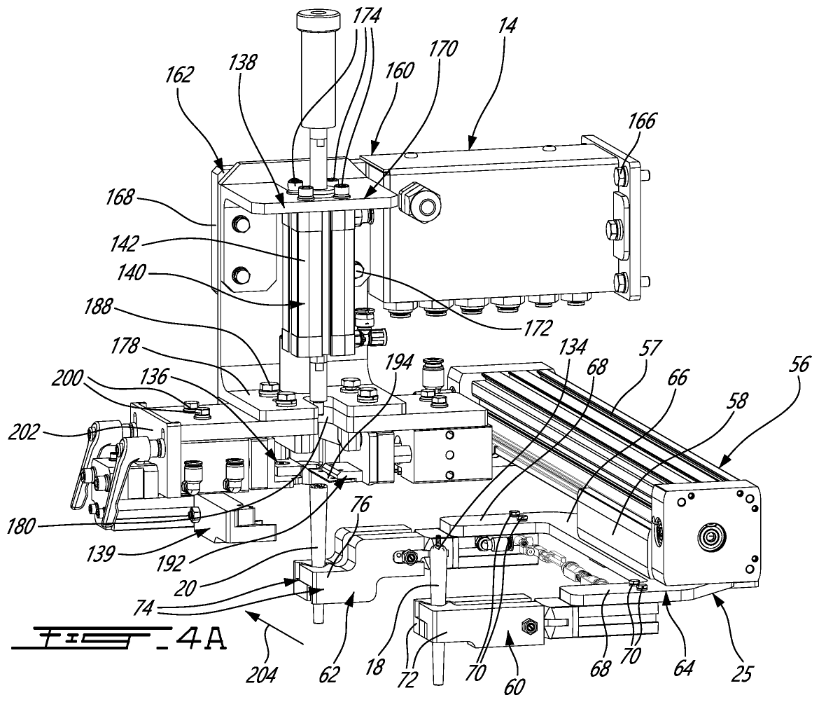

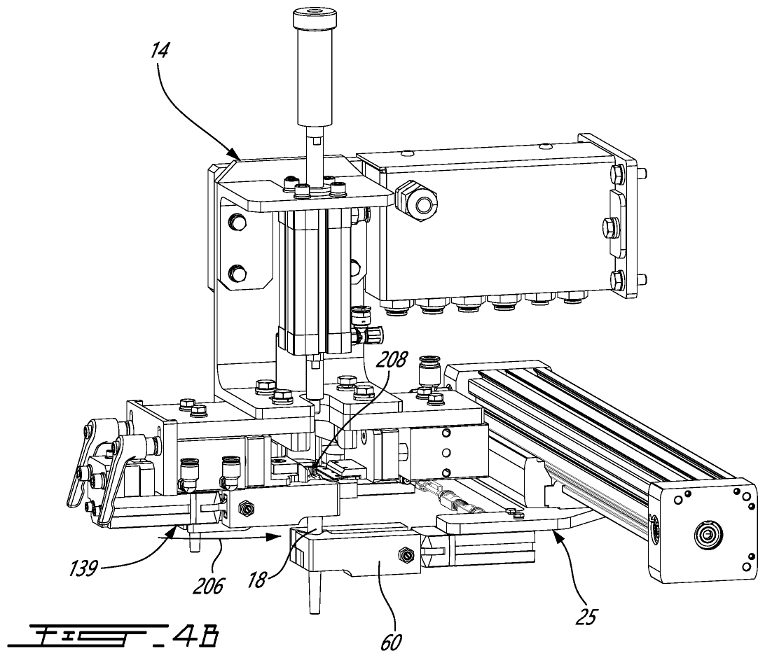

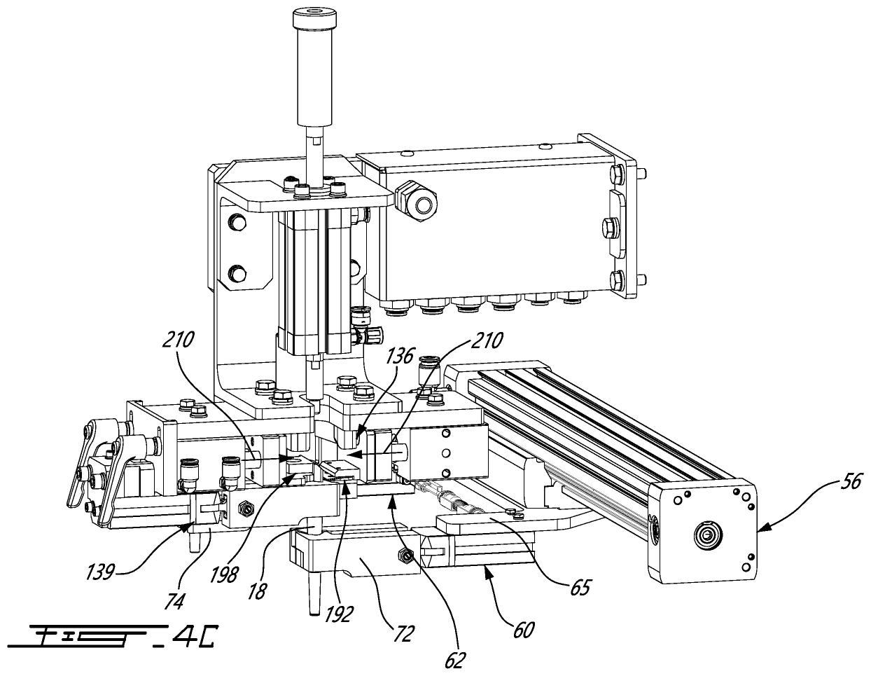

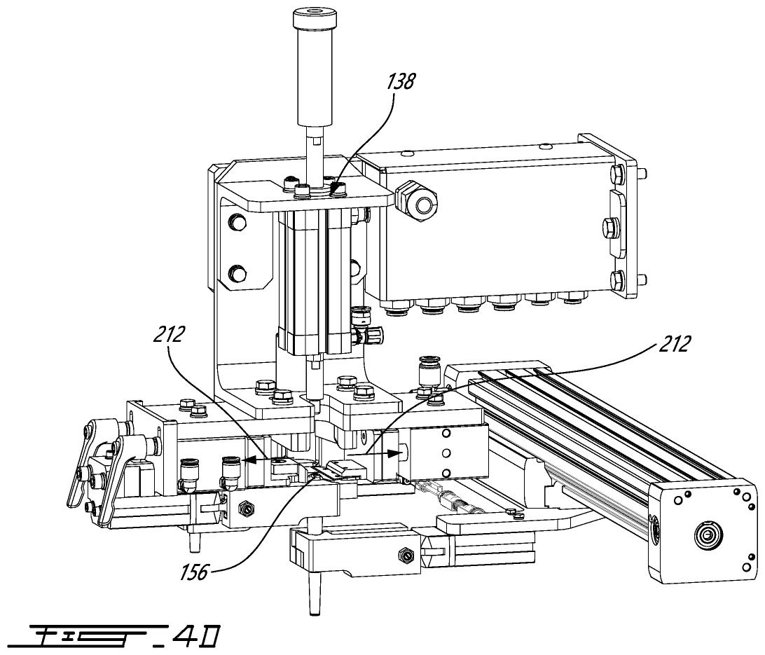

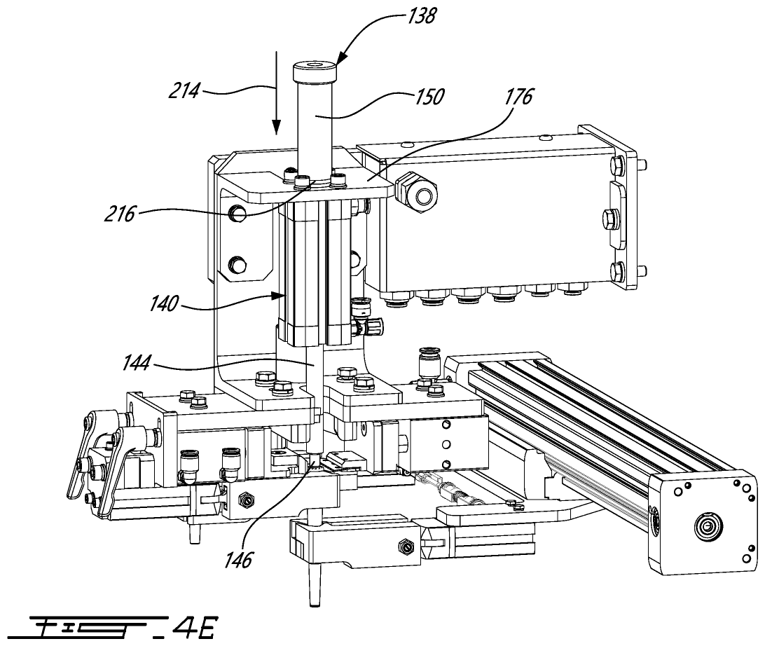

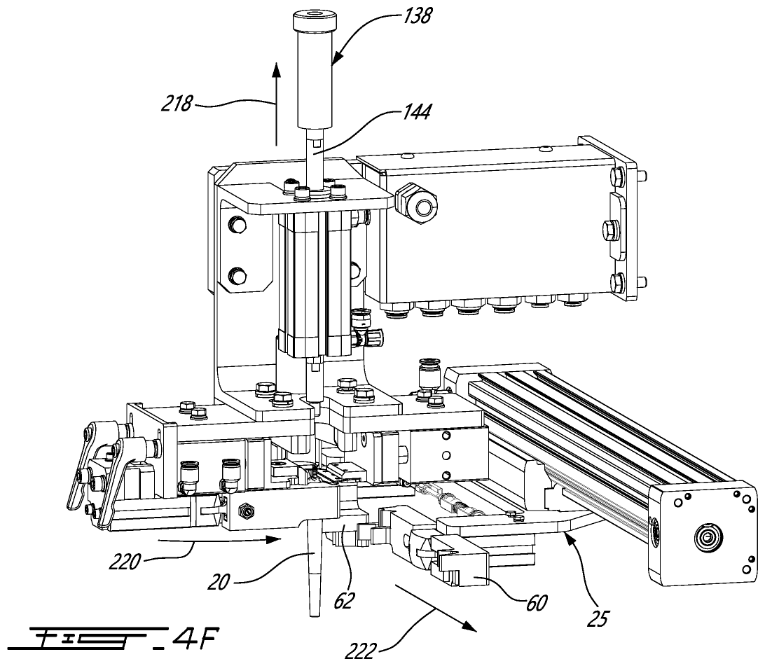

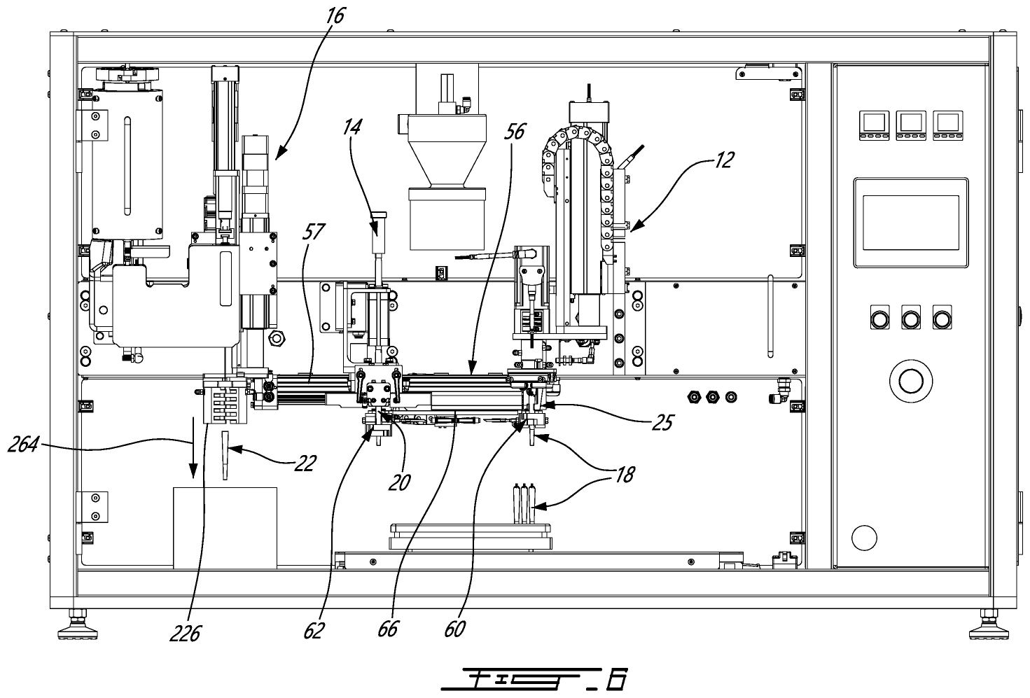

With references to FIGS. 4A and 6, the cannabis cone moving device 25 will now be described in more detail.

The cannabis cone moving device 25 comprises a linear actuator 56, having a base 57 and a slider 58 conventionally mounted to the base 57 for sliding movements therealong, and first and second grippers 60-62 mounted to the slider 58 for sliding movement along the base 57 in unison with the slider 58.

The first and second grippers 60-62 are fixedly mounted to the slider 58 via a U-shaped plate 64. More specifically, the U-shaped plate 64 is defined by a central portion 66 secured to the slider 58 thereunder using fasteners (not shown), and two lateral arms 68 integrally extending from the central portion 66 on opposite lateral side thereof. Each of the first and second grippers 60-62 are secured to a respective arm 68 of the U-shaped plate 64 thereunder using fasteners 70 so that both grippers 60-62 extend substantially perpendicularly from the base 57 for gripping cannabis cones 18-20 and products 22 oriented substantially perpendicular to both the base 57 and grippers 60-62.

The grippers 60 and 62 have respective pairs of gripping jaws 72 and 74 that are shaped for complementary receiving a cannabis cone 18-20 or product 22 therein. While the gripping jaws 72 of the first gripper 60 are substantially straight, the gripping jaws 74 of the second gripper 62 are generally S-shaped, yielding a gripping portion 76 that is lower than the first gripper 60, for a reason that will be explained hereinbelow.

Both grippers 60 and 62 are movable between a cannabis cone holding position, wherein both their gripping jaws 72 and 74 respectively are generally parallel and joined (see for example on FIG. 4A), and a cannabis cone releasing position, wherein their respective jaws 72 and 74 are opened and extends substantially perpendicular to the arms 68 of the U-shaped plate 64 on respective side thereof (see on FIG. 4F).

The skilled person will now appreciate that, while in their cone-releasing position, the grippers 60 and 62 minimize their dimension in the direction perpendicular to the base 57 of the actuator 56, maximizing clearance between the base 57 and the stations 12-16, thereby facilitating their displacement along the stations 12-16.

While the grippers 60 and 62 are configured for gripping cannabis supports in the form of a cannabis cones 18-20 and products in the form of cannabis joints 22, grippers according to another illustrative embodiment (not shown) are configured for gripping cannabis supports and products having another form.

Also, the linear actuator 56 is not limited to the illustrated embodiment and may take any other form allowing to controllably move the grippers 60-62 from stations 12-16 to stations 12-16.

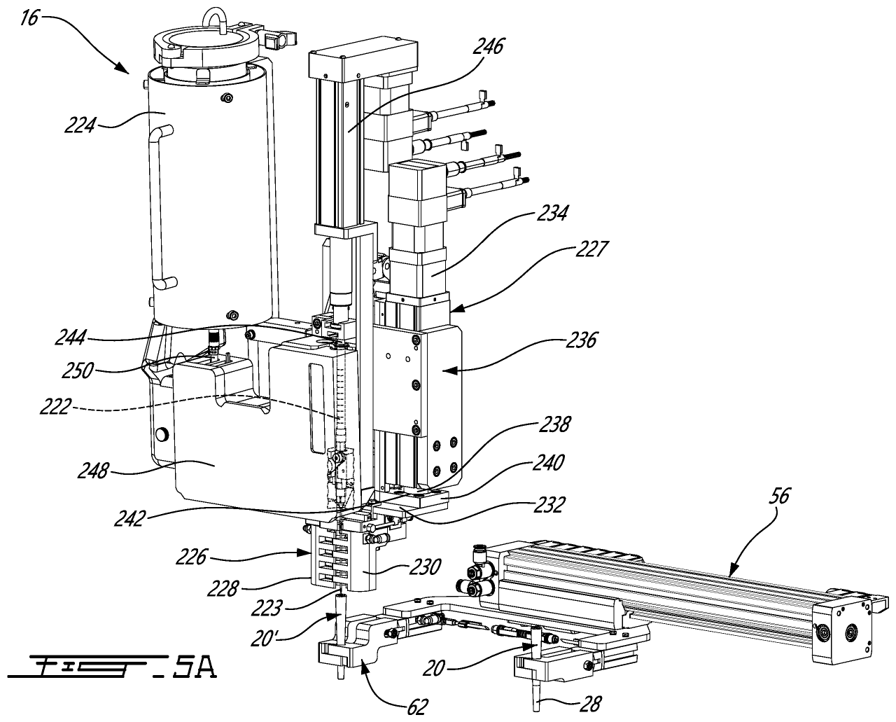

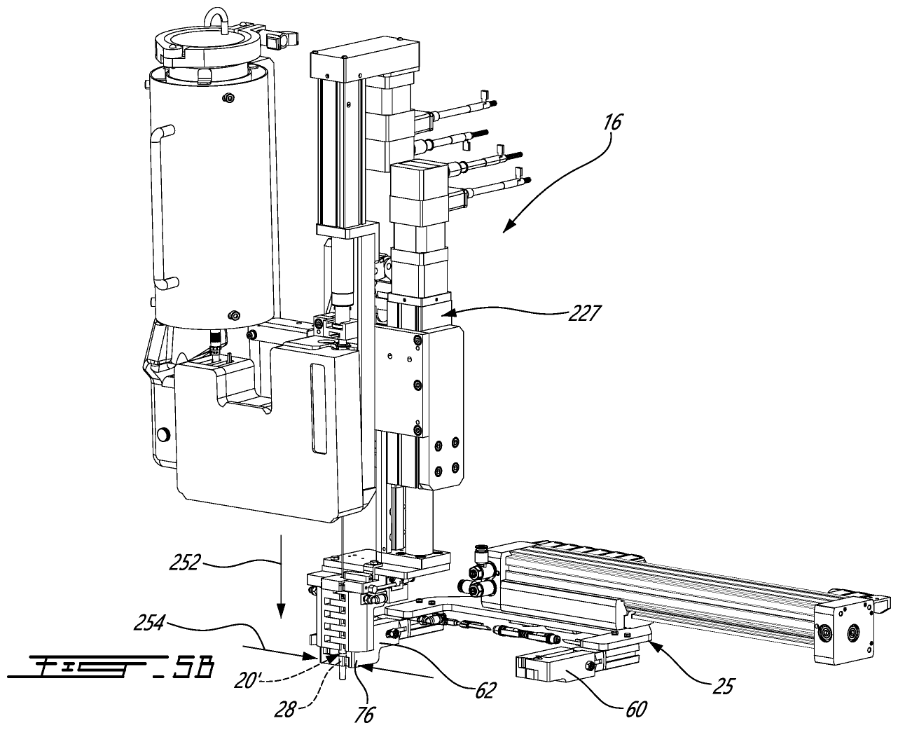

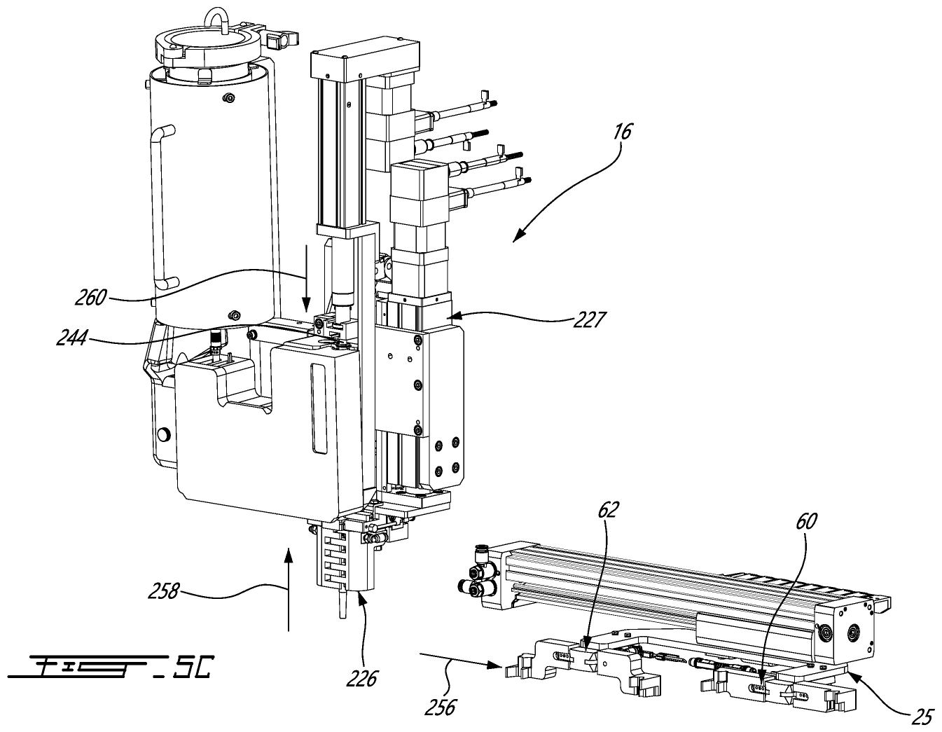

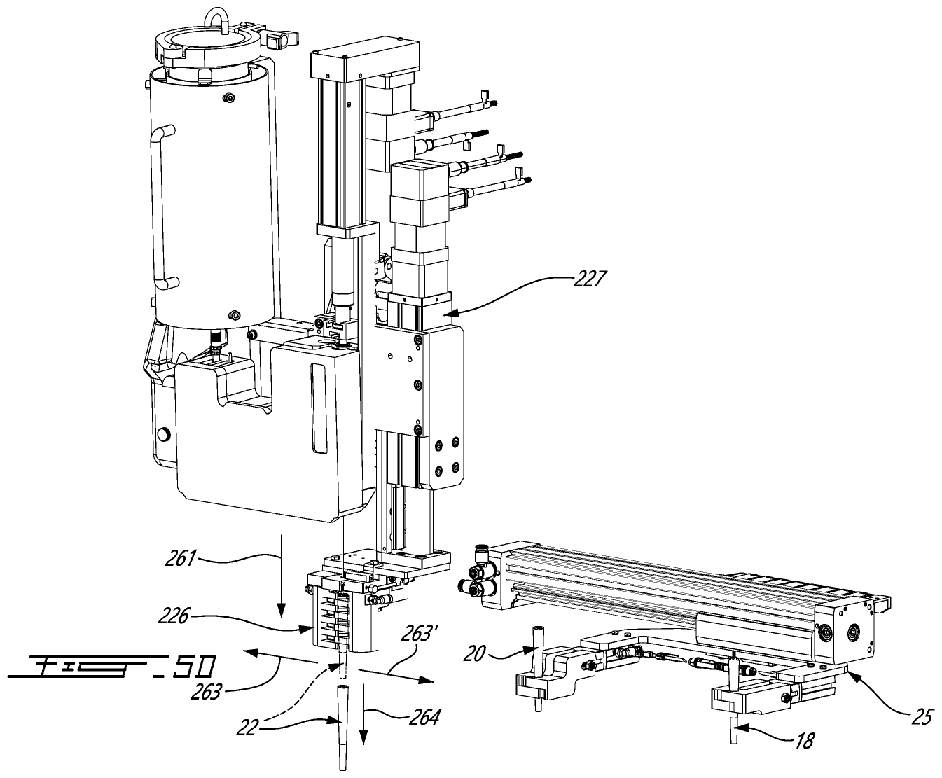

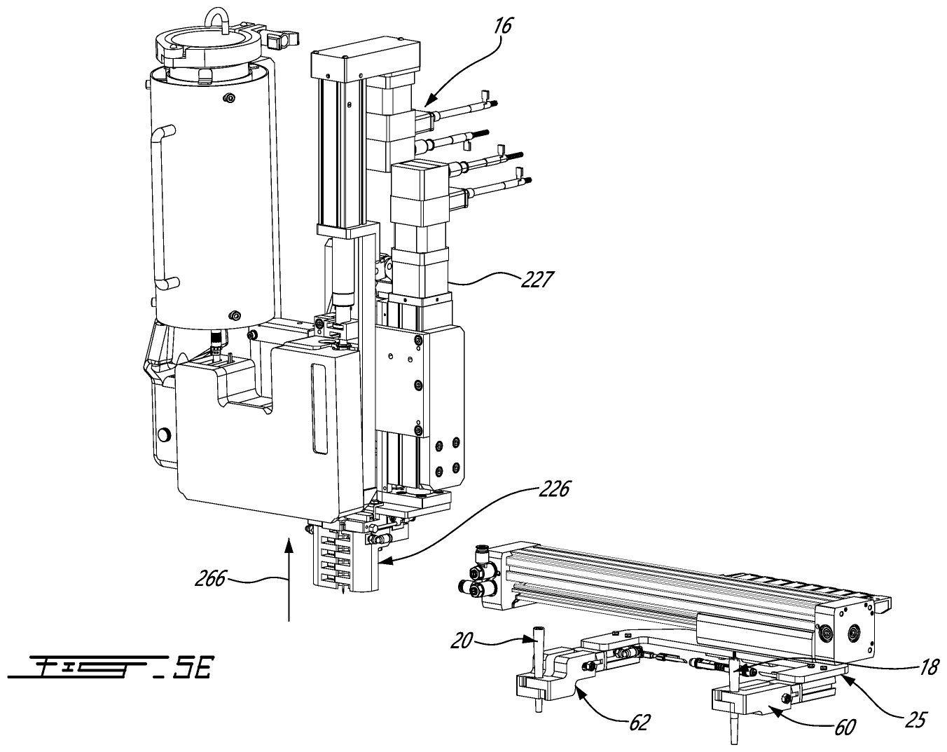

As can be seen in comparing FIGS. 6 and 5A, the grippers 60 and 62 are so mounted to the linear actuator 56, and the length of the linear actuator 56 is such that:
i) the end of the course of the slider 58 on the base 57 at its outmost right position causes the operational alignment of the first gripper 60 with the cannabis cone picking station 12, and of the second gripper 62 with the cannabis cone forming station 14, and
ii) the end of the course of the slider 58 on the base 57 to its outmost left position causes the operational alignment of the first gripper 60 with the cannabis cone forming station 14, and of the second gripper 62 cannabis oil injecting station 16.

Further characteristics of the cannabis joints moving device 25 will become more apparent further in with reference to further detail on its operation thereof.

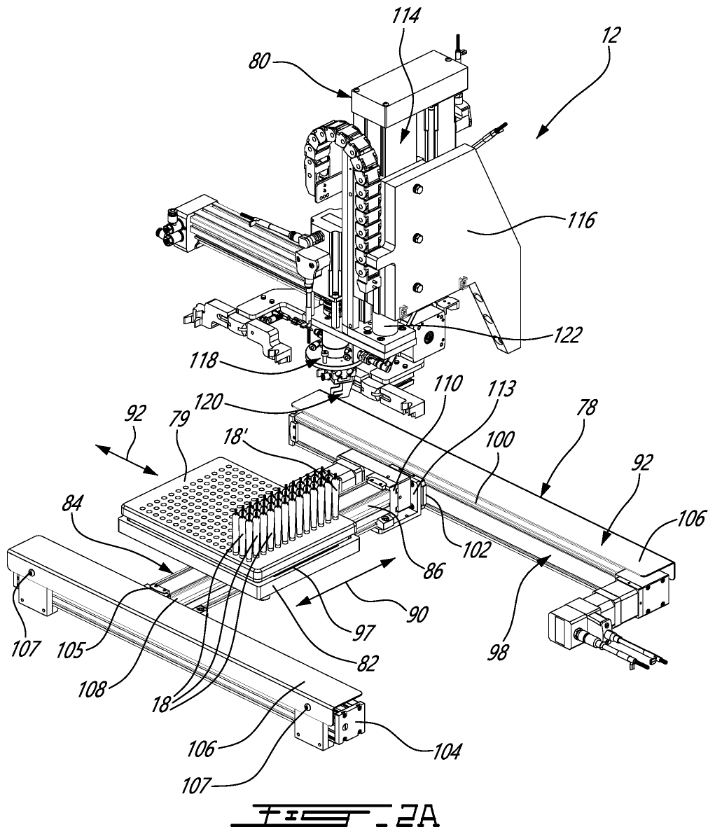

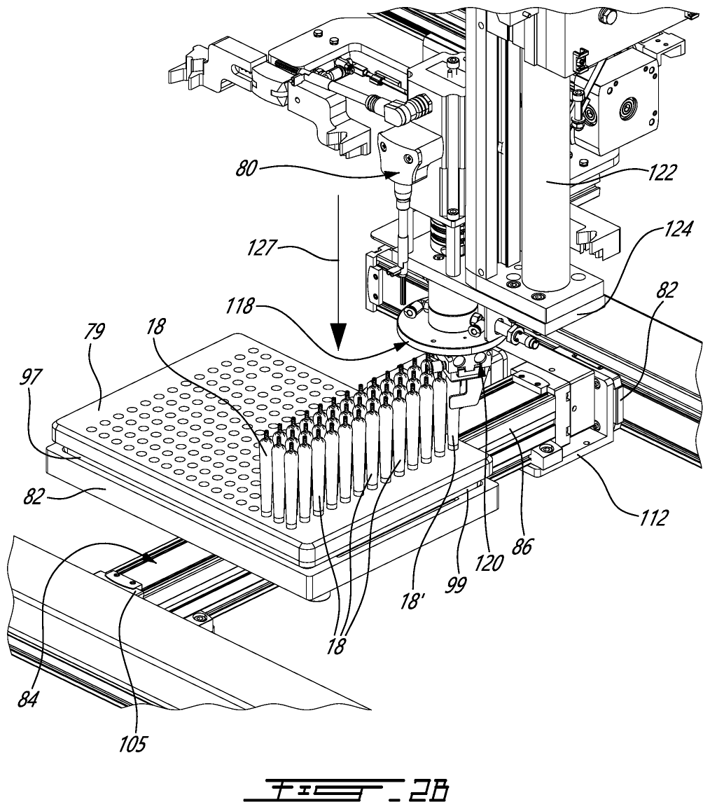

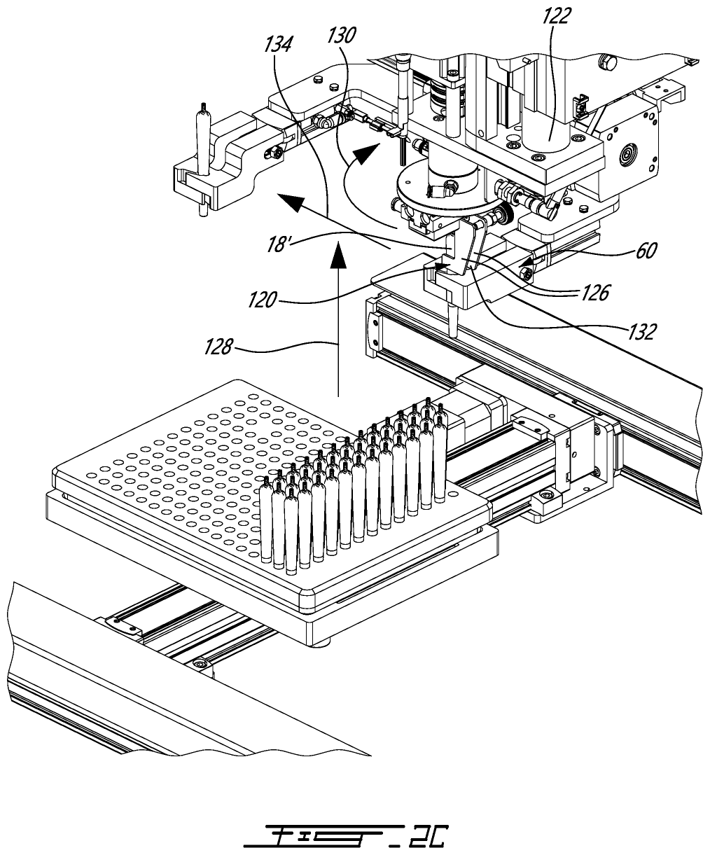

Turning now to FIG. 1-2C, the cannabis cone picking station 12 will now be described in more detail.

The station 12 comprises a cannabis tray-receiving and moving device 78 that is configured for receiving a tray 79 of cannabis cones 18 and a cannabis cone picking device 80 for picking a cannabis cone 18 therefrom.

The cannabis tray-receiving and moving device 78 comprises a first linear actuating mechanism 84, having a base 86 and a slider (not shown), conventionally mounted to base 86 for sliding movement along a first axis (see arrows 90), that fixedly receives a tray-receiving table 82, and a second linear actuating mechanism 92 that receives the first linear actuating mechanism 84 for reciprocal movement along a second axis perpendicular to the first axis (see arrow 92), rending the tray-receiving table movable in two dimensions.

The tray-receiving table 82 includes a rectangular opening 96 for complementary rectangular-shaped protrusion 97 under the tray 79. According to another illustrative embodiment, the table 82 is provided with other attachment parts or mechanisms (not shown) that are adapted to fixedly receive the tray 79 or a tray having another configuration (not shown) so that the tray 79 can move in unison with the table 82.

The tray-receiving table 82 is attached to the slider of the actuating mechanism 84 for solidary movements therewith using fasteners or other well-known fastening means or method. According to another illustrative embodiment, the tray-receiving table 82 is integral to the slider.

The second linear actuating mechanism 92 includes a linear actuator 98, having a base 100 and a slider 102, a first sliding member in the form of a track 104 positioned in front of the linear actuator 98, parallel thereto having a second sliding member, in the form of cursor 105 mounted thereto, for reciprocating movement therealong (see arrow 92). Each of the base 100 and track 104 are secured respectively to the back and front wall 38 and 42 of the casing 26, via an L-shaped bracket 106. Fasteners 107 (only two shown) or other attachments are used for securing the base 100 and track 104 to the bracket 106 and the bracket 106 to the bottom wall 36.

The longitudinal ends 108 and 110 or the base 86 of the first actuating mechanism 92 are secured to the cursor 105 and slider 102 respectively via an L-shaped bracket 112.

In operation of the cannabis tray-receiving and moving device 78, both actuating mechanisms 84 and 92 are precisely controlled to position the tray-receiving table 82, and therefore any tray 79 of cones 18 thereon, so that a specific cone 18 is appropriately positioned to be picked by the cannabis cone picking device 80. Since the configuration of the tray 79 and the positions of the cones 18 therein is provided to the controller (not shown) of the cannabis tray-receiving and moving device 78, the position of the cannabis cones 18 are indexed and the cannabis tray-receiving and moving device 78 can be operated for their precise displacement relative to the picking device 80.

The cannabis cone picking device 80 comprises a rod-style actuator 114, that is secured to a mounting plate 115 on the back wall 38 via a mounting bracket 116, a rotary actuator 118 mounted to the linear actuator 114, and a small gripper 120 mounted to the rotary actuator 118.

The rotary actuator 118 is secured to the sliding rod 122 of the rod-style actuator 114 via a mounting plate 124.

The small gripper 120 is secured to the output of the rotary actuator 118 and includes a pair of generally L-shaped jaws 126 that are movable between open and close positions, wherein a cannabis cone 18 can be held between the jaws 126 while in their close position (see FIG. 2C).

The operation of the cannabis cone picking station 12 will now be described with reference to FIGS. 2A-2C.

The rotary actuator 118 of the cannabis cone picking device 80 is first actuated to pivot the small gripper 120 so that the opening side of the jaws 126 are aligned with the direction that the next available cone 18′ that will be presented thereto (see FIG. 2A). It is to be noted that the jaws 126 are then in their opened position.

The rod-style actuator 114 is then operated to push its rod 122 to a position where the gripper 120 is operationally levelled with the upper portion of the cones 18 in the tray 79 (see arrow 127 in FIG. 2B).

The cannabis tray-receiving and moving device 78 is then actuated as described hereinabove so that the next available cannabis cone 18 in the tray 79 is moved within the jaws 126 of the gripper 120 of the cannabis cone picking device 80. As described hereinabove, the positions of the cone 18 in the tray 79 is indexed relative to the gripper 120. More specifically, the tray 79 is moved (see arrows 90 and 92) to position the next available cone 18′ from the left of the gripper 120, in front thereof (see FIG. 2B).

The gripper 120 is then actuated to move in its close position, where its jaws 126 precisely close onto the presented cone 18′.

With reference to FIG. 2C, the rod-style actuator 114 is then operated to retract its rod 122 to move the cone 18′ in the gripper 120 in position to be picked up by the first gripper 60 of the cannabis cone moving device 25 (see arrow 128 in FIG. 2C), simultaneously to the operating the rotary actuator 118 to pivot the small gripper 120 to its initial orientation (see arrow 130). According to this orientation, the opening 132 of the jaws 126 is aligned with the next moving direction of the grippers 60-62 (see arrow 134).

In a next step of the system 10, the first gripper 60 is closed onto the cannabis cone 18′ and is then moves the cannabis cone 18′ to the next station 14 (see arrow 134).

This operational cycle of the station 12 is repeated for each further cannabis cone 18 in the tray 79. Of course, this cycle is synchronized with the operation of the other stations 14-16.

In an embodiment wherein the inputs of the system 10 is a cannabis cone similar to the cone 18, but wherein the top end thereof is open, and therefore not twisted, a person skilled in the art will appreciate that the rotary actuator 118 can be further used to rotate the top, while it is squeezed by the gripper 120, to form a twisted tip as illustrated.

The first and second linear actuating mechanisms 84 and 92 are not limited to the first illustrative embodiment and can take other forms allowing to move the table 82 within a plane.

Also, the table may be adapted to receive other cannabis cone support than the illustrated tray 79. The cannabis cone picking device 80 is not limited to the illustrative embodiment and may take another form allowing to pick cannabis cones or other cannabis supports.

Also, the sequence of operation of the station 12 may be different than illustrated and described.

The cannabis cone forming station 14 will now be described in more detail with references first to FIGS. 3 and 4A.

According to the first illustrative embodiment, the cannabis cone forming station 14 receives as input from the cannabis cone picking station 12, a cannabis cone 18 including cannabis therein, the cone including a filter 28 at its bottom end, and a twisted top 134 at its top end. As can be seen in FIG. 4A, the output of the station 14 is the same cannabis cone 18, but with its twisted top 134 cut and flatted, which is referred to as cannabis cone 20. According to another illustrative embodiment (not shown), the forming station 14 receives as input a cannabis cone 18 having an untwisted top end (not shown).

The cannabis cone forming station 14 comprises a tip cuter 136 and a tip flattener 138 and a cannabis cone gripper 139.

The tip flattener 138 comprises a rod-style actuator 140, having a base 142, and a rod 144 operatively mounted therein, an impact tip 146, secured to the rod 144 at the bottom end 148 thereof, and a course stopper 150, secured at the upper end 152 thereof.

The impact tip 146 has an impacting portion 154 that has diameter slightly less than the diameter of the top portion 156 of the cannabis cone 18 and an integral connecting portion 158 that is mounted to the rod 144. According to the first illustrative embodiment, the bottom end 148 of the rod 144 and the connecting portion 158 are configured for complementary connection, for example by including complementary threaded portions. According to another illustrative embodiment, the rod the impact tip 146 is integral to the rod 144. According to another illustrative embodiment, the end of the tip 146 is pointed, allowing to pre-piercing of the cone 18 from the top, to ease the further insertion of the injection needle in the next station.

The course stopper 150 is secured to the upper end 152 of the rod 144 similarly to the mounting of the impact tip 146 to the rod 144. According to another illustrative embodiment (not shown) one or both of the impact tip 146 and course stopper are mounted to the rod 144 so that their distance to the base 142 can be adjusted.

The base 142 is secured to the back wall 38 of the casing 26 via a mounting assembly 160. The mounting assembly 160 includes a first L-shaped bracket 162, having a first flat portion 164 secured to the wall 38 using fasteners 166, and a second elongated flat portion 168 extending perpendicularly from the first portion 164. The mounting assembly 160 further includes a U-shaped bracket 170, secured to the L-shaped bracket using fasteners 172. The actuator 140 is secured to the U-shaped bracket 170 using fasteners 174. For that purpose, the top wall 176 of the bracket 170 is provided with an aperture (not shown) to allow passage for the rod 144, and the bottom wall 178 of the bracket 170 includes a notch 180 to allow passage for the impact tip 146 therethrough.

The tip cuter 136 comprises first and second rod-style actuators 182, each having a base 184, a piston rod 186 that is operatively mounted to the base 184 therein, and a flat head 188 integrally mounted to the piston rod 186 at the distal end 190 thereof.

The tip cutter 136 further comprises a cutter 192, including a blade 194, that is secured to one of the flat head 188 via an L-shaped blade support 196, and a stopper 198, that is secured to the other flat head 188 so as to come into contact with the blade 194 when the tip cutter 136 is actuated.

Each of the L-shaped blade support 196 and stopper 198 are secured to the corresponding head 188 using fasteners (not shown) or welding for example.

Each of the first and second rod-style actuators 182 are secured to the bottom wall 178 of the bracket 170 thereunder on opposite sides of the notch 180 via a mounting plate 185 using fasteners 200 and 201.

The cannabis cone gripper 139 is secured to the one of the two actuators 182 via a mounting bracket 202 and is movable between an open position (shown in FIG. 4A) and a close position (shown in FIG. 3) where it is in position to grip a cannabis cone 18 brought by the second gripper 62.

The operation of the cannabis cone forming station 14 will now be described with references to FIGS. 4A-4F.

FIG. 4A shows the moving device 25 with one ‘pre-formed’ cannabis cones 18 in its first gripper 60 and one formed cannabis cone 20 in its second gripper 62. The cannabis cone 20 has just been formed by the station 14, which is then ready to receive the pre-formed cannabis cone 18, which is then in the station 12.

In the next step, the cannabis cone moving device 25 is actuated to move both grippers 60-62 one step to the left (see arrow 204) moving each of the cannabis cones 18 and 20 respectively to stations 14 and 16.

FIG. 4B shows the cannabis cone 18 in station 14, wherein the gripper 139 of the station 14 just closed onto the cannabis cone 18 (see arrow 206), while the first gripper 60 of the moving device 25 still holding the cannabis cone 18. At that time the cannabis cone 20 is arrived at the station 16.

Turning now to FIG. 4C, while both grippers 139 and 60 still holding the cannabis cone 18, the tip cuter 136 is actuated so that both the cutter 192 and stopper 198 are moved towards one another to cut the tip 208 of the cannabis cone 18 (see arrows 210).

As shown in FIG. 4D, the tip cuter 136 is then moved to its open position (see arrows 212), showing a cannabis cone 18 that is now without tip 208, clearing the way for the operation of the tip flattener 138.

The operation of the flattener 138 will now be described with references to FIGS. 4E-4F.

In operation of the tip flattener 138, the actuator 140 is operated to move the rod 144 downwardly (see arrow 214) until the impact tip 146 flatten the top portion 156 of the cannabis cone 18. The penetration distance of the impact tip 146 beyond the upper portion of the cannabis cone 18 is limited by the position of the course stopper 150, which comes into contact with the top wall 176 of the U-shaped bracket 170, and more specifically with washers 216 installed on the wall 176 around the rod 144 to minimize wear on the wall 176.

According to another illustrative embodiment, such penetration distance of the impact tip 146 is further or alternatively determined by a controlled operation of the actuator 140.

FIG. 4F shows the retraction of the rod 144 (see arrow 218) and the opening of the gripper 60 (see arrow 220), before the moving device 25 is actuated to move both grippers 60 and 62 to the previous stations 12 and 14 respectively. The second gripper 62 will then close onto the formed cannabis cone 20 before the gripper 139 of the station 14 releases its grip onto the cone 20 (see FIG. 4A). A new cycle of the station 14 can then begin.

According to another illustrative embodiment (not shown), the cannabis cone forming station 14 includes only one of the tip flattener 138 and cannabis cone gripper 139 and or another cannabis support forming mechanism. Also, other mechanisms than illustrated may be used to cut the tip 208 of a cannabis cone 18 or flat its top portion 156.

The cannabis oil injecting station 16 will now be described in more detail with reference first to FIG. 5A.

The cannabis oil injecting station 16 comprises a syringe 222 having a needle 223, a cannabis oil reservoir 224 that is operatively coupled to the syringe 222 and that is configured to feed a predetermined single dose of cannabis oil thereto, a cone gripper 226 for holding a cannabis cone 20 presented by the second gripper 62 of the cannabis cone mover 25, and a linear actuator 227 for moving the cone gripper 226 and the cannabis cone 20 in the gripper 226 so that the cone 20 is penetrated by the injection needle 223 of the syringe 222.

The gripper 226 comprises a pair of claws 228-230 that are mounted to a mounting plate 232 for sliding movement towards and away each other via a motorized rack and pinion or another similar mechanism (not shown). The pair of claws 228-230 are shaped for complementary receiving the upper portion of a cannabis cone 20 when they are closed. According to another illustrative embodiment, the grippers 226 takes another form than illustrated. According to still another embodiment, the gripper 226 is replaced by another mechanism allowing to take and support the cannabis cone 20.

The linear actuator 227 includes a base 234, secured to the back wall 38 of the casing 26 via a mounting assembly 236, a shaft 238 operatively mounted in the base 234, and a rectangular plate 240 secured at the distal end 242 of the shaft 238.

The gripper 226 is so mounted to the actuator 227 via the plates 232 and 240 that i) the opening defined by the claws 228-230 when they are closed, ii) the needle 223 of the syringe 222, and iii) the second gripper 62 are all operationally aligned when the second gripper 62 is moved to the station 16.

The syringe 222 includes a plunger 244 that is operatively coupled to a linear actuator 246 for its actuation. The actuator 246 and syringe 222 are so secured to the mounting assembly 236 as to provide the above-mentioned alignment.

The cannabis oil injecting station 16 further includes a heated casing 248 that is configured to heat tubing 250 between the oil reservoir 224 and the needle 223 and therefore the oil therein to a predetermined temperature, so that the physical properties of the oil, such as its viscosity, remain, even when the distribution of oil to the syringe 223 is stopped.

The expression ‘cannabis oil’ should be construed herein and in the claim as including, without limitations, CBD (cannabidiol), THC (Tetrahydrocannabinol), terpenes, cannabis resin or other component in oil form.

The heated casing 248 includes elements or another heat-generating device or mechanism.

The operation of the cannabis oil injecting station 16 will now be described with references to FIGS. 5A-5E.

FIG. 5A shows the station 16 a new cannabis cone 20′, following its forming in the previous station 14. The cone 20′ is held by the second gripper 62 and positioned registered with the gripper 226 and needle 223, which is extends from the opening center of the gripper 226. The gripper 226 is then in its open configuration.

The gripper 226 is longer than the grippers 60-62 mainly to yield a better grip on the cannabis cone 20′ during its injection. This has been found to prevent the cannabis cone 20′ from being damaged during injection, and to maintain in place the cone 20′, thereby forcing the course of the needle 223 centered therethrough.

The actuator 227 is then activated to lower the gripper 226 so that it positioned on the portion of the cannabis cone 20′ above the filter (see arrow 252 in FIG. 5B) while the second gripper 62 of the mover 25 still holding the cannabis cone 20′ by its lower filter portion 28. A person skilled in the art will now appreciate that second gripper 62 having a gripping portion 76 that is lower than the first gripper 60 allows holding the cannabis cone 20′ by its lower portion 28, while allowing clearance for the gripper 226 of the station 16 to hold simultaneously the upper portion of the cone 20′.

When the gripper 226 is positioned for gripping the cone 20′, it is actuated to close thereon (see arrows 254). The grippers 60-62 then open.

With references to FIG. 5C, the moving device 25 is then actuated to move back both grippers 60-62 to the previous stations 12 and 14 respectively (see arrows 256). According to the first illustrative embodiment, this is done simultaneously to the actuator 227 being activated to slowly raise the gripper 226 with the cone 20′ (see arrow 258).

During the insertion of the needle 223, the operation of the plunger 244 is synchronized, via the actuator 246 (see arrow 260), with the insertion of the needle 223 in the cannabis cone 20′. This allows to better spread the cannabis oil in the cannabis cone 20′

As described hereinabove, after it has been injected with cannabis oil, the cannabis cone 20′ is referred to herein as the cannabis joint 22.

With references to FIG. 5D, the actuator 227 is activated to lower the gripper 226 (see arrow 261) while it still holds the cannabis joint 22 (in dashed line in FIG. 5D).

The gripper 226 then opens (see arrows 263, 263′) to release the resulting product 22, which falls in a bin 262 (see on FIG. 1) under the gripper 226 (see arrows 264 in FIGS. 1 and 5D).

With references to FIG. 5E, the actuator 227 is operated to raise the empty gripper 226, which is then ready for a new cycle

During these steps (shown in Figured 5D and 5E), the first and second grippers 60 and 62 of the cannabis cone moving device 25 are registered respectively in stations 12 and 14, each holding cannabis cones 18 and 20 respectively, ready to be moved in the next stations 14 and 16 (see FIG. 6).

It is to be noted that the cannabis oil injecting station 16 is not limited to the illustrated embodiment. For example, the gripper 226 can be modified or replaced by another cannabis product holding mechanism (not shown). The relative positions of the syringe 222, needle 223 and or reservoir 224 relative to the gripper 226 or to one another can be different than illustrated. Anyone or both linear actuators 227 and 246 can be replaced by another mechanism allowing to move the gripper 226 and/or syringe 222.

According to another illustrative embodiment, the station 16 is configured so that the syringe 222 is moved towards the gripper 226, instead of the opposite.

Since actuators and grippers and their operation are believed to be well-known in the art, they will not be described herein in more detail for concision purposes.

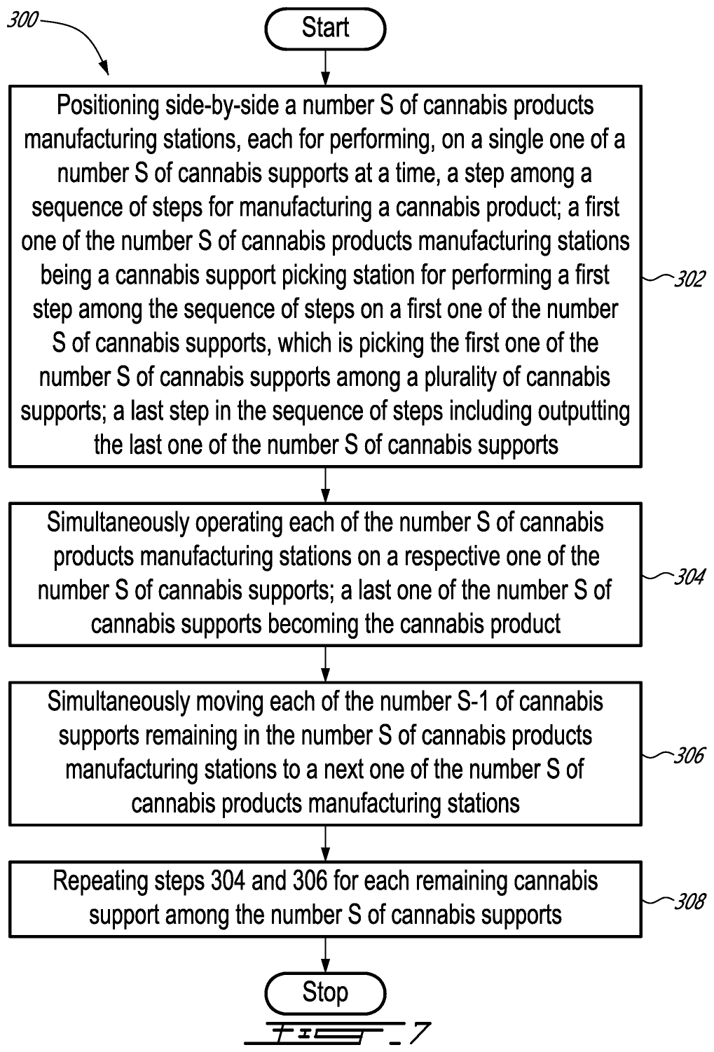

The overall operation of the system 10 is summarized in FIG. 7, which illustrates a process for manufacturing cannabis, in the form of cannabis joints 22, wherein the cannabis supports are in the form of cannabis cones.

It is to be noted that the actuators described hereinabove are connected to one or more of the control modules described hereinabove, so that their operations are triggered and synchronized thereby. All the connectors between such controller(s) and the actuators have been omitted in the drawings to alleviate the views.

While the actuators mentioned hereinabove and illustrated in the drawings are of the pneumatic type, they can also be of another type, including without limitations hydraulic or electric.

It is to be noted that many modifications could be made to the illustrative system 10 and process 300 described hereinabove and illustrated in the appended drawings. For example:
the sequence of operations of the system 10, including the timing of use of the cannabis support moving device 25 between steps performed by the manufacturing stations 12-16, may be different than illustrated and described;
while the cannabis supports 18-22 have been described as including cannabis cones, each provided with a filter 28 and including cannabis therein, they can have other forms, such as cannabis rolled in paper (not shown). According to some embodiment (not shown), the filter 28 is omitted;
the cannabis support provided to the cannabis joints manufacturing system can alternatively be empty cannabis cones (not shown) and one of the manufacturing stations includes a cone filling device (not shown). According to still another illustrative embodiment, the cannabis support provided are cannabis cone filled with cannabis with an open top end. According to this particular illustrative embodiment, the manufacturing stations includes a cannabis cone top twister (not shown) that yields a closed cannabis cone 18;
the final product 22 can also be different than illustrated, depending on both the initial cannabis support form and the functions of the manufacturing stations;
while the system 10 is illustrated as including a bin 262 for receiving the final products 22, other product-collecting system can be provided, such as a chute, a conveyor, etc. or any combination thereof;
the order, number and/or functions of the cannabis support transforming stations may be different than illustrated;
while the system 10 has been illustrated to output cannabis joints 20 having their tip 28 cut, their top portion 256 flattened and cannabis oil injecting therein, it can be modified to result in another output;
the configuration, size and number of the mounting elements may be different than illustrated;
the assembly of components of the system 10, using for example fasteners, brackets, or mounting assemblies, may be replaced by permanent assembly, using, for example welding.

Although a system and process for manufacturing cannabis products have been described hereinabove by way of illustrated embodiments thereof, they can be modified. It is therefore to be understood that numerous modifications may be made to the illustrative embodiments and that the scope of the claims should not be limited by the preferred embodiment but should be given the broadest interpretation consistent with the description as a whole.

What is claimed is:

1. A cannabis products manufacturing system comprising:
a number S of at least two different cannabis products manufacturing stations, each for performing on one cannabis support at a time a step among a sequence of steps for manufacturing a cannabis product; the number S of at least two different cannabis products manufacturing stations being positioned side-by-side according to the sequence of steps for manufacturing the cannabis product; a first one of the cannabis products manufacturing stations being a cannabis support picking station for performing a first step among the sequence of steps, which is picking said one cannabis support among a plurality of cannabis supports; and
a cannabis support moving device, including a number S minus 1 of cannabis support grippers arranged side-by-side so that each of the cannabis support grippers is operatively paired with a corresponding one of the manufacturing stations for simultaneous reciprocal movement between the corresponding one of the manufacturing stations and a next one thereof; each of the cannabis support grippers being movable between a cannabis support holding position and a cannabis support releasing position.

2. The system as recited in claim 1, wherein the plurality of cannabis supports are cannabis cones.

3. The system as recited in claim 1, wherein the cannabis product is a cannabis joint.

4. The system as recited in claim 1, wherein the number S is 3.

5. The system as recited in claim 1, wherein the number S of at least two different cannabis products manufacturing stations are selected from the group consisting of a station for forming said one cannabis support and a station for injecting oil in said one cannabis support.

6. The system as recited in claim 5, wherein said one cannabis support is a cone of cannabis closed by a twisted tip; the station for forming the cannabis support includes a tip cutter for cutting the twisted tip, yielding a cone of cannabis with a rounded end.

7. The system as recited in claim 6, wherein the station for forming the cannabis support further includes a flattener for flattening the rounded end of the cone of cannabis.

8. The system as recited in claim 7, wherein the tip cutter defines an opening when it is opened; the flattener including an impact tip that is movable through the opening.

9. The system as recited in claim 7, wherein the station for forming the cannabis support further includes a gripping device for gripping the cone of cannabis from one of the number S minus 1 of cannabis support grippers of the cannabis support moving device.

10. The system as recited in claim 5, wherein said one cannabis support is a cone of cannabis having a distal end that is closed; wherein the station for forming the cannabis support includes a flattener for flattening the closed distal end.

11. The system as recited in claim 10, wherein the flattener includes a rod-style actuator, having a base and a rod operatively mounted to the base therein, an impact tip, secured to the rod at a bottom end thereof, and a rod course stopper, secured at the upper end of the rod.

12. The system as recited in claim 5, wherein said one cannabis support is a cone of cannabis; the station for injecting cannabis oil includes i) an oil reservoir, ii) a syringe connected to the oil reservoir for receiving oil therefrom and having a needle and a plunger, ii) an actuator for operating the plunger and iii) a gripping device for gripping the cone of cannabis from one of the number S minus 1 of cannabis support grippers of the cannabis support moving device; the gripping device being movable towards and away the needle.

13. The system as recited in claim 1, wherein the number S of at least two different cannabis products manufacturing stations and the cannabis support moving device are comprised in a casing.

14. The system as recited in claim 1, wherein the cannabis support moving device includes a linear actuator comprising a base and a slider slidably mounted to the base; the number S minus 1 of cannabis support grippers being mounted to the slider.

15. The system as recited in claim 14, wherein each of the number S minus 1 of cannabis support grippers has a pair of jaws that are movable between a cannabis support holding position and a cannabis support releasing position; the cannabis support releasing position yielding the pairs of jaws substantially parallel to the base.

16. The system as recited in claim 1, wherein at least one of the number S minus 1 of cannabis support grippers has a pair of jaws that are both generally S-shaped.

17. The system as recited in claim 1, wherein at least one of the number S minus 1 of cannabis support grippers has a gripping portion configured for complementary receiving a cannabis cone.

18. The system as recited in claim 1, wherein the cannabis support picking station includes a device for receiving and moving a tray including the plurality of cannabis supports therein and a device for picking on the tray said one cannabis support among the plurality of cannabis supports.

19. The system as recited in claim 18, wherein the device for receiving and moving a tray includes a first linear actuating mechanism, having a first base and a first slider, a table configured for receiving the tray secured to the first slider, and a second linear actuating mechanism, having a second base and a second slider; the first base being secured to the second slider for solidary movement therewith.

20. The system as recited in claim 18, wherein the device for picking on the tray said one cannabis support among the plurality of cannabis supports includes i) a rod-style actuator having a rod, ii) a rotary actuator mounted to the rod for solidary linear movement therewith and having an output, and iii) a small gripper secured to the output of the rotary actuator for solidary movement therewith for said picking on the tray said one cannabis support among the plurality of cannabis supports.

21. The system as recited in claim 18, wherein said picking a single cannabis support among a plurality of cannabis supports is performed during the simultaneous reciprocal movement of the cannabis support grippers between the corresponding one of the manufacturing stations and a next one thereof.

22. A cannabis products manufacturing system comprising:
a number S of at least two different cannabis joints manufacturing stations, each for performing on one cannabis cone at a time a step among a sequence of steps for manufacturing a cannabis joint; the at least two different cannabis joints manufacturing stations being positioned side-by-side according to the sequence of steps for manufacturing the cannabis joint; a first one of the cannabis joints manufacturing stations being a cannabis cone picking station for performing a first step among the sequence of steps, which is picking said one cannabis cone among a plurality of cannabis cones; and
a cannabis cone moving device, including a number S minus 1 of cannabis cone grippers arranged side-by-side so that each of the cannabis cone grippers is operatively paired with a corresponding one of the manufacturing stations for simultaneous reciprocal movement between the corresponding one of the manufacturing stations and a next one thereof; each of the cannabis cone grippers being movable between a cannabis cone holding position and a cannabis cone releasing position; wherein the cannabis support moving device includes a linear actuator comprising a base and a slider slidably mounted to the base; the number S minus 1 of cannabis support grippers being mounted to the slider;
wherein the number S of at least two different cannabis joints manufacturing stations are selected from the group consisting of a cannabis joint forming station and a cannabis oil injecting station.

23. A process for manufacturing cannabis products, the process comprising:
i) positioning side-by-side a number S of cannabis products manufacturing stations, each for performing, on a single one of a number S of cannabis supports at a time, a step among a sequence of steps for manufacturing a cannabis product; a first one of the number S of cannabis products manufacturing stations being a cannabis support picking station for performing a first step among the sequence of steps on a first one of the number S of cannabis supports, which is picking a first one of the number S of cannabis supports among a plurality of cannabis supports; a last step in the sequence of steps including outputting a last one of the number S of cannabis supports;
ii) simultaneously operating each of the number S of cannabis products manufacturing stations on a respective one of the number S of cannabis supports; the last one of the number S of cannabis supports becoming the cannabis product; and
iii) simultaneously moving each of a number S-1 of cannabis supports remaining in the number S of cannabis products manufacturing stations to a next one of the number S of cannabis products manufacturing stations;
iv) repeating ii) and iii) for each remaining cannabis support among the number S of cannabis supports.

24. The process as recited in claim 23, wherein the number S of cannabis products manufacturing stations are selected from the group consisting of a station for forming the cannabis support and a station for injecting oil in the cannabis support.

25. The process as recited in claim 23, wherein the cannabis supports are cannabis cones.

26. The process as recited in claim 23, wherein the cannabis product is a cannabis joint.

27. The process as recited in claim 23, wherein the number S is 3.

28. The process as recited in claim 23, wherein the cannabis support picking station includes a first linear actuating mechanism, having a first base and a first slider, a table configured for receiving the tray secured to the slider, and a second linear actuating mechanism, having a second base and a second slider; the first base being secured to the second slider for solidary movement therewith.

29. The process as recited in claim 23, wherein the cannabis support picking station includes a) a rod-style actuator having a rod, b) a rotary actuator mounted to the rod for solidary linear movement therewith and having an output, and c) a small gripper secured to the output of the rotary actuator for solidary movement therewith for said picking the first one of the number S of cannabis supports among a plurality of cannabis supports.

30. The process as recited in claim 23, wherein said picking a single cannabis support among a plurality of cannabis supports is performed during said iii) simultaneously moving each of a number S-1 of cannabis supports remaining in the number S of cannabis products manufacturing stations to a next one of the number S of cannabis products manufacturing stations.

31. A cannabis joints manufacturing machine comprising:
a plurality of cannabis joints manufacturing devices positioned in a line; a first one of the plurality of cannabis joints manufacturing devices receiving a cannabis cone including cannabis therein; each of the other one of the plurality of cannabis joints manufacturing devices being adapted to perform on the cannabis cone at least one step among a sequence of steps towards manufacturing a cannabis joint; a last one of the plurality of cannabis joints manufacturing devices transforming a cannabis cone therein into a cannabis joint and outputting the cannabis joint; and
a cannabis cone moving device for simultaneously moving the cannabis cones in the plurality of cannabis joints manufacturing devices to a next cannabis joints manufacturing device in the line.
